(19) United States
(12) Patent Application Publication
(10) Pub. No.: US 2018/0325741 A1
(43) Pub. Date: Nov. 15, 2018

(54) BIODEGRADABLE NEGATIVE PRESSURE WOUND THERAPY DRESSING

(71) Applicants: BAXTER INTERNATIONAL INC., Deerfield, IL (US); BAXTER HEALTHCARE S.A., Glattpark (Opfikon) (CH)

(72) Inventors: Kevin Michael Lewis, Burlington, WI (US); Adam R. Marek, Arlington Heights, IL (US); Shawn F. Bairstow, Gurnee, IL (US)

(21) Appl. No.: 15/979,823

(22) Filed: May 15, 2018

Related U.S. Application Data
(60) Provisional application No. 62/506,592, filed on May 15, 2017.

(51) Int. Cl. A61F 13/00; A61M 1/00; A61L 15/32; A61L 15/42

(52) U.S. Cl. CPC A61F 13/00012; A61M 1/0088; A61F 13/00021; A61F 2013/00174; A61L 15/425; A61F 13/00995; A61F 13/00063; A61L 15/32

(57) ABSTRACT

The disclosure provides methods of treating a wound using negative pressure wound therapy by contacting a wound with a porous fibrin foam dressing and applying negative pressure on the wound, wherein the fibrin foam includes a three-dimensional reaction product of fibrinogen and thrombin and is biodegradable in vivo. Further provided is a dressing material for negative pressure wound therapy comprising a porous fibrin foam that can substantially conform to a shape of the wound, wherein the fibrin foam includes a three-dimensional reaction product of fibrinogen and thrombin, is biodegradable in vivo, and does not need to be changed during the course of therapy.

### FIELD OF THE DISCLOSURE

The disclosure relates generally to a wound dressing for negative pressure wound therapy. More particularly, the disclosure relates to a method of treating a wound using negative pressure wound therapy including a fibrin foam dressing material.

### BACKGROUND

Wound care is a worldwide clinical issue with a significant economic impact, affecting millions of patients, both chronically and acutely, each year. To treat and close wounds, a number of options are available, including the traditional use of sutures, staples, gauze and tapes, as well as modern therapies such as surgical sealants and glues, hydrogels, foams, alginates, thermal/energy-based wound closures, and negative pressure wound therapy. These products and procedures are designed to facilitate at least one stage of the multiphase wound healing process, generally consisting of four main stages: hemostasis, inflammation, proliferation, and remodeling.

Negative pressure wound therapy (NPWT) has evolved as a clinical treatment due to the beneficial effects it has on the healing of both chronic and acute wounds. The therapy applies a sub-atmospheric pressure to the local wound environment using a sealed wound dressing connected to a vacuum pump. NPWT has been shown to promote healing through the drainage of excess fluids, contraction of the perimeter of the wound, reduction of tissue edema, and mechanical stimulation of the wound bed giving rise to angiogenesis and the formation of granulation tissue. Additionally, NPWT can reduce the risk of infection by decreasing the formation of bacteria in the wound, as the wound is sealed and protected.

In conventional NPWT, typically the wound is filled with a polyurethane (PU) foam or gauze dressing in order to occupy the dead space known to impede wound closure. Filling the dead space of the wound prevents fluid from accumulating, and thereby reduces the chance of infection, as well as provides a bridge for new tissues to spread across the wound surface. The wound is then sealed with a semi-permeable dressing which is connected to a vacuum pump. Negative pressure is continuously applied to the wound at a constant pressure, for example between about −200 mm Hg and about −75 mm Hg, such as −125 mm Hg. Every two to five days, or at the clinician's discretion, the dressing must be removed and replaced to prevent bacterial infection and for assessment of wound healing. When the wound dressing is changed, the foam or gauze dressing must also be replaced. Through this process, the healthy granulation tissue that had grown into the dressing during the treatment must be cut and removed, hindering wound healing and causing pain for the patient.

As noted, the wound dressing used in NPWT is typically a PU foam or gauze. Gauze dressings induce the formation of granulation tissue that is stable, but thin. Furthermore, gauze dressings do not promote hypertrophic tissue growth. In contrast, PU foam has been shown to induce the formation of thick, yet fragile, granulation tissue, which can result in scarring upon healing. PU foam intentionally has large pore sizes (400-600 microns) in order to facilitate capillary action in the dressing when vacuum pressure is applied, so as to assist in the removal of wound exudate and minimize the chance of infection.

Another modern treatment for wound healing is the use of fibrin sealants. Fibrin sealants are composed of a mixture of fibrinogen and thrombin. Fibrin sealants are used in skin graft fixation as a replacement or adjunct to staples and sutures as they are designed for use as a glue, or tissue adhesive. They are used in surgery to promote hemostasis and tissue sealing. Due to their small pore size, they are not compatible with NPWT.

### SUMMARY

This disclosure provides a method of treating a wound using NPWT including contacting the wound with a porous fibrin foam dressing and applying negative pressure on the wound, wherein the fibrin foam comprises a three-dimensional reaction product of fibrinogen and thrombin and is biodegradable in vivo.

In a related aspect, the disclosure provides a dressing material for NPWT, including a porous fibrin foam that can substantially conform to a shape of the wound, wherein the fibrin foam comprises a three-dimensional reaction product of fibrinogen and thrombin, is biodegradable in vivo, and does not need to be changed during the course of therapy.

In a related aspect, the disclosure provides a method of treating a wound using NPWT including contacting the wound with a porous foam dressing and applying negative pressure on the wound, wherein the porous foam comprises pores characterized by a mean pore size in a range of about 75 microns to about 300 microns, and the foam is biodegradable in vivo.

Further aspects of the disclosure may become apparent to those skilled in the art from a review of the following detailed description, taken in conjunction with the appended claims. While the invention is susceptible of embodiments in various forms, described herein are specific embodiments of the invention with the understanding that the disclosure is illustrative, and is not intended to limit the invention to specific embodiments described herein.

### BRIEF DESCRIPTION OF THE DRAWINGS

FIG. 1 shows a prior art mixing hand-held device.

FIG. 2 shows the pore size distribution of fibrin foam prepared by 6 passes of 4 IU/ml thrombin solution and 91 mg/ml fibrinogen solution through a mixing device in the presence of gas.

FIGS. 3A-3E show images of a murine wound model before application of four different wound dressings (FIG. 3A) and after removal of the dressings at 3d (FIG. 3B), 7d (FIG. 3C), 10d (FIG. 3D), and 14d (FIG. 3E).

FIGS. 4A-4B show the change in mean wound size as a percent of initial wound size for each dressing in a murine wound model after 7-days (FIG. 4A) and after 14-days (FIG. 4B).

FIG. 5 shows histopathological analyses of a murine wound model after application of the wound dressings for 7-days and 14-days.

FIGS. 6A-6B show a fibrin foam dressing in a porcine wound model after direct application of negative pressure to the wound.

FIGS. 7A-7B show a prior art V.A.C.® Freedom NPWT System (FIG. 7A) and procedure for NPWT (FIG. 7B).

FIGS. 8A-8C show an untreated lesion (FIG. 8A) that is then treated with fibrin foam (FIG. 8B) and the same lesion treated with fibrin foam after a period of 48 hours in which NPWT was applied, wherein cellular debris is centralized and removed by NPWT (FIG. 8C).

### DETAILED DESCRIPTION

The disclosure provides a method of treating a wound using NPWT with a biodegradable dressing including a porous fibrin foam, wherein the fibrin foam comprises a three-dimensional reaction product of a gas, fibrinogen and thrombin. The disclosure further provides a method of treating a wound using NPWT including contacting the wound with a porous, biodegradable, foam dressing and applying negative pressure on the wound, wherein the porous foam comprises pores characterized by a mean pore size in a range of about 75 microns to about 300 microns Advantageously, the foams of all aspects disclosed herein are biodegradable in vivo, thus do not need to be removed or changed throughout the course of treatment and can remain in contact with the wound until the foam biodegrades. In some cases, the foam disclosed herein can biodegrade over the course of fourteen days. Advantageously, the foam disclosed herein can be characterized by pore size and porosities that facilitate angiogenesis and cell growth into the foam, can substantially conform to the shape of the wound, and/or can be applied to inverted or vertical surfaces.

The fibrin foam of the disclosure can be prepared from a fibrinogen solution, a thrombin solution, and gas. The fibrinogen solution concentration, thrombin solution concentration, and amount of gas are not particularly limiting. The fibrin foam of the disclosure can be prepared from a volume of fibrinogen solution having a concentration in a range of about 1 mg/ml to about 200 mg/ml, about 25 mg/ml to about 150 mg/ml, about 50 mg/ml to about 150 mg/ml, about 75 mg/ml to about 125 mg/ml, about 80 mg/ml to about 120 mg/ml, or about 90 mg/ml to about 110 mg/ml, for example, about 200 mg/ml, about 150 mg/ml, about 125 mg/ml, about 100 mg/ml, about 75 mg/ml, or about 50 mg/ml. The volume of thrombin solution can have a concentration in a range of about 0.01 IU/ml to about 10,000 IU/ml, about 0.01 IU/ml to about 1,200 IU/ml, about 0.01 IU/ml to about 800 IU/ml, about 0.1 IU/ml to about 500 IU/ml, about 500 IU/ml to about 700 IU/ml, about 800 IU/ml to about 1200 IU/ml, about 0.05 IU/ml to about 400 IU/ml, about 0.1 IU/ml to about 250 IU/ml, about 0.5 IU/ml to about 100 IU/ml, about 1 IU/ml to about 50 IU/ml, or about 4 IU/ml to about 10 IU/ml, for example, about 8 IU/ml, about 6 IU/ml, about 4 IU/ml, about 2 IU/ml, or about 1 IU/ml. In embodiments, the fibrin foam of the disclosure can be prepared from a volume of fibrinogen solution having a concentration of about 100 mg/ml and a volume of thrombin solution having a concentration of about 4 IU/ml. The ratio of the volume of the fibrinogen solution to the volume of the thrombin solution can be about 3:1 to about 1:3, about 3:1 to about 1:2, about 2:1 to about 1:3, about 2:1 to about 1:2, or about 1:1. The volume of gas to the sum of the volume of fibrinogen solution and thrombin solution can be in a range of about 1:4 to about 4:1, about 1:3 to about 3:1, about 1:2.5 to about 3:1, about 1:1 to about 2.5:1, or about 1:1.25 to about 1.25:1. The gas can be any gas suitable for preparing a medicament and applying said medicament to a wound. For example, the gas can comprise nitrogen, oxygen, or combinations thereof, such as air. In the absence of a gas, the resulting mixture of fibrinogen solution and thrombin solution does not form a foam. Rather, the resulting mixture of fibrinogen solution and thrombin solution forms a liquid fibrin sealant which, when set, provides a relatively non-porous fibrin material. A relatively non-porous material includes little or no pores and the pores that are included have pore sizes of 25 microns or less.

The disclosure further provides a method to prepare the fibrin foam using a device to combine gas, fibrinogen, and thrombin with the use of a three-dimensional lattice defining a plurality of tortuous interconnecting passages. In embodiments, the device includes at least one mixing disc having two opposing sides and comprising a three-dimensional lattice defining a plurality of tortuous, interconnecting passages therethrough. The mixing disc having two opposing sides and comprising a three-dimensional lattice defining a plurality of tortuous, interconnecting passages therethrough may be referred to herein as a “mixing disc.” A first container in fluid communication with one side of the mixing disc holds a fibrinogen solution, while a second container in fluid communication with the other side of the mixing disc holds a thrombin solution. Each container is in fluid communication with the other container through the mixing disc to allow one of the fibrinogen solution or thrombin solution to flow from one side of the mixing disc to the other side of the mixing disc and to allow return flow of both components through a mixing disc. Furthermore, at least one of the first container or second container further comprises the volume of gas. The concentration and volumes of the fibrinogen solution and thrombin solution and the volume of gas can be any concentration and/or volume as described herein.

The mixing disc is made of a porous material and may have varying porosity depending on the application. Such porous material preferably has a porosity that allows the streams of the components to pass through to create a thoroughly-mixed combined fluid stream. The porosity of a material may be expressed as a percentage ratio of the void volume to the total volume of the material. The porosity of a material may be selected depending on several factors including but not limited to the material employed and its resistance to fluid flow (creation of excessive back pressure due to flow resistance should normally be avoided), the viscosity and other characteristics and number of mixing components employed, the quality of mixing that is desired, and the desired application and/or work surface. By way of example and not limitation, the porosity of a material that may be employed for mixing may be between about 20% and 60%, preferably between about 20% to 50% and more preferably between about 20% and 40%.

Also, the mean pore size range of the mixing disc may vary. The mixing disc may define a plurality a pores that define at least a portion of the flow paths through which the streams of the components flow. The range of mean pore sizes may be selected to avoid undue resistance to fluid flow of such component streams. Further, the mean pore size range may vary depending on several factors including those discussed above relative to porosity. Several mean pore size ranges for different materials for the mixing disc are shown in Table 1, except at no. 16 which includes a “control” example that lacks a mixing disc.

TABLE 1
PART III: Evaluation of mixing discs
Materials from Porvent and Porex

| Sample ID | Type | Form | Property | Mean Pore Size | | Thickness | Mixing |
|---|---|---|---|---|---|---|---|
| 2 | | PE sheet | Hydrophobic | 5-55 | μm | 2.0 mm | good |
| 21 | | PP sheet | Hydrophobic | 15->300 | μm | 2.0 mm | good |
| 6 | | PE sheet | Hydrophobic | 20-60 | μm | 3.0 mm | Good |
| 19 | | PP sheet | Hydrophobic | 70-210 | μm | 1.5 mm | Good |
| 22 | | PP sheet | Hydrophobic | 70-140 | μm | 3.0 mm | Good |
| 24 | | PP sheet | Hydrophobic | 125-175 | μm | 3.0 mm | Good |
| 1 | | | Hydrophobic | 7-12 | μm | 1.5 mm | no fibrin |
| | | | | | | | extrusion |
| 8 | | PE sheet | Hydrophobic | 40-90 | μm | 1.5 mm | Good |
| 7 | | PE sheet | Hydrophobic | 20-60 | μm | 1.5 mm | Good |
| 9 | | PE sheet | Hydrophobic | 20-60 | μm | 3.0 mm | Good |
| 16 | | PE sheet | Hydrophobic | 40-100 | μm | 1.5 mm | Good |
| 18 | | PE sheet | Hydrophobic | 40-100 | μm | 3.0 mm | Good |
| 20 | | PE sheet | Hydrophobic | 80-130 | μm | 3.0 mm | Good |
| 14 | | PE sheet | Hydrophobic | 20-60 | μm | 1.5 mm | Good |
| 17 | | PE sheet | Hydrophobic | 80-130 | μm | 1.5 mm | Good |
| 26 | Control | — | — | — | | — | — |
| 27 | | PP sheet | Hydrophobic | 7-145 | μm | 1.5 mm | Good |

Table 1 includes several commercial sintered polyethylene (PE) or polypropylene (PP) materials manufactured by Porex or by Porvair under the tradename PORVENT or VYON. The table summarizes the mixing results achieved from each material based on quality of fibrin obtained after fibrinogen and thrombin (4 International Units (IU)/ml) passed through a device having a single mixing disc, except for one experiment (sample ID 26) which is the control and does not include mixing disc. The indicated mean pore size of the mixing disc ranges vary between about 5 and 300 microns. In Table 1, the ranges for materials nos. 2, 21, 6, 19, 22, 24, 8-9, 16, 18, 20, 14, 17, and 27 each generally indicate good mixing quality for fibrin. In Table 1, such mean pore size ranges are not intended to be exhaustive and other mean pore size ranges are also possible and useful for mixing. The mean pore size ranges indicated in Table 1 were obtained from the technical data sheets of the listed materials provided by the suppliers Porvair and Porex.

The mixing disc may be further configured and sized so as to provide sufficiently thorough mixing of the streams of the components. The size of the mixing disc may vary depending on such factors which include the size and/or configuration of the dispenser, the mixing disc porosity and mean pore size, the mixing disc material employed, the desired degree of mixing, the mixing components, and/or the desired application. For a mixing disc having the above discussed example ranges for porosity and mean pore sizes, the mixing disc thickness may range between about 1.5 mm and 3.0 mm, as indicated in Table 1. Other thicknesses are also possible including a variable or non-uniform thickness.

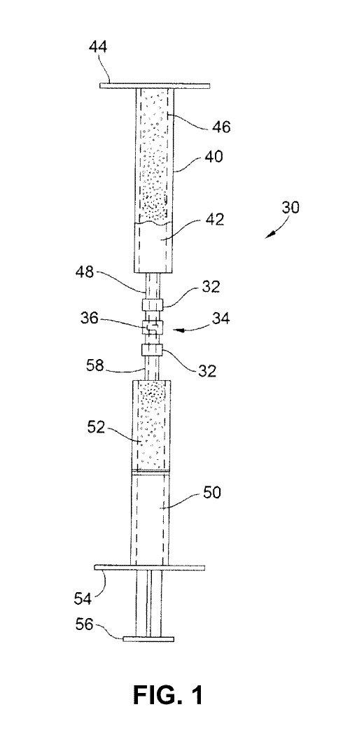

Methods of making fibrin foam are known in the art. For example, U.S. Pat. No. 8,512,740 B2, the disclosure of which is herein incorporated by reference in its entirety, describes compositions of fibrin foam prepared by injecting a pre-prepared fibrinogen foam into a thrombin solution using a mixing device, as shown in FIG. 1. The fibrinogen foam can be made using the mixing technique described, with one syringe including 0.5 ml fibrinogen, of a concentration from about 1 mg/ml to about 110 mg/ml, and the other syringe including 1.0 ml gas. As an example, the mixing device prepares a foam from the fibrinogen solution and gas by combining the fibrinogen solution and gas and then passing the combination through a mixing disc comprising a sintered polymeric material forming a three-dimensional lattice defining a plurality of tortuous interconnecting passages. The fibrinogen foam is then injected into a solution of thrombin, of a concentration from about 0.01 IU/ml to 500 IU/ml, again using the mixing device to produce the desired fibrin foam. The mixing device prepares a fibrin foam from the fibrinogen foam and thrombin solution by combining the fibrinogen foam and thrombin solution and then passing the combination through the mixing disc comprising the sintered polymeric material forming a three-dimensional lattice defining a plurality of tortuous interconnecting passages.

In the present disclosure, the mixing method described by U.S. Pat. No. 8,512,740 B2 has been modified to allow the fibrinogen solution and thrombin solution to admix in the presence of gas through a three-dimensional lattice defining plurality of tortuous, interconnecting passages therethrough. Similar to the mixing device described in U.S. Patent Application No. 2009/0038701 A1, the disclosure of which is herein incorporated by reference in its entirety, the mixing procedure of the present disclosure can be achieved by using two containers holding one of each of the thrombin and fibrinogen solutions. Additionally, at least one of these containers contains the volume of gas.

The ratios of the components used to make the fibrin foam can vary, depending on the viscosity or firmness of foam desired. In general, as the concentration of thrombin is increased relative to the concentration of fibrinogen, the setting time of the fibrin foam decreases and more force may be needed to apply the fibrin foam from the mixing device. Increasing the concentrations of the thrombin solution can result in very firm foams that cannot be applied through the tip of the mixing device, as the setting of the foam occurs too rapidly. The fibrin foam instead takes the shape of its container, which it maintains after removal from the container. This allows the user to select the shape of the resulting fibrin foam. For example, for a very firm fibrin foam, an increased thrombin solution concentration of about 250 to 500 IU/ml can be used with a fibrinogen solution concentration of about 1 mg/ml to about 100 mg/ml. For a more formable or malleable foam, a lower concentration of thrombin solution, from about 4 IU/ml to 20 IU/ml, can be used with a fibrinogen solution concentration of about 1 mg/ml to about 100 mg/ml.

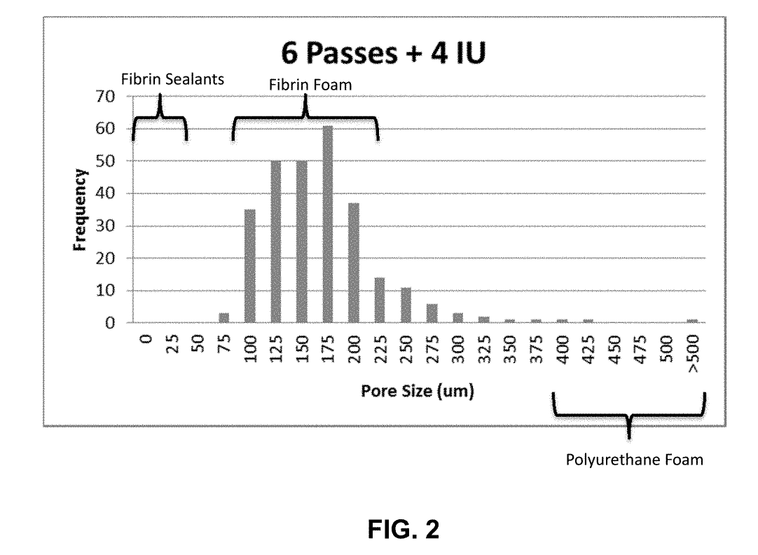

The number of passes of fibrinogen solution and thrombin solution through the device can affect the quantity and size of the pores of the resultant fibrin foam, as can be seen in the FIG. 2. Without intending to be bound by theory, it is believed that the pore size is dependent on both the number of passes made through the mixing device, as well as the concentration of the thrombin solution used. Thus, the number of passes chosen can vary depending on the desired pore size of the fibrin foam. In general, fewer number of passes results in larger pore sizes, while more passes results in smaller pore sizes. In embodiments, the fibrin foam can reach its foam-like consistency and appearance within about 20 seconds after about 6 passes through the mixing device. The number of passes can be in a range of about 2 to about 25 passes, about 2 to about 20 passes, about 4 to about 15 passes, about 10 to about 15 passes, about 4 to about 10 passes, about 5 to about 8 passes, or about 5 to about 7 passes. As used herein, a “pass” constitutes movement of the fibrinogen solution, thrombin solution and/or a mixture thereof from the first or second container through the mixing disc and into the other of second or first container. Thus, the initial movement of thrombin solution and/or fibrinogen solution through the mixing disc to provide a fibrinogen/thrombin mixture constitutes one pass. Thus, a second pass will transfer the mixed solution through the mixing disc and into the other respective container. In embodiments, the fibrin foam can be applied about 15 seconds to about 2 minutes after completing the passes through the mixing device, such as about 30 seconds to about 1 minute, about 15 seconds to about 30 seconds, within 15 seconds, within 20 seconds, within 30 seconds, within 1 minute, and/or within 2 minutes after the passes through the mixing device. The firmness of the foam can vary depending on the amount of time that elapses after completing the passes through the mixing device and before application of the foam, with firmer foams generally being formed after longer hold times.

The fibrin foam of the disclosure is a porous and three-dimensional reaction product of gas, fibrinogen and thrombin. The pores of the fibrin foam are characterized by a mean pore size in a range of about 75 microns to about 300 microns, about 75 microns to about 200 microns, or about 100 microns to about 200 microns, for example, at least about 75 microns, at least about 100 microns, at least about 125 microns, at least about 150 microns, or at least about 175 microns, and up to about 300 microns, up to about 275 microns, up to about 250 microns, up to about 225 microns, or up to about 300 microns. The porosity of the fibrin foam is at least 50% and up to about 95%, or at least about 60% and up to about 80%, for example, at least 50%, at least about 60%, at least about 65%, or at least about 70% and up to about 95%, up to about 90%, up to about 85%, or up to about 80%. In general, it is desirable that the fibrin foam has as many pores as possible of the appropriate pore size in order to facilitate wound healing and removal of wound exudate. In embodiments, the fibrin foam has a mean pore size of about 100 microns to 200 microns, for example, about 155.5±58.3 microns and a porosity of about 60% to about 80%, for example about 72.5±8.3%.

The porous foam of the disclosure can include any material that is biodegradable in vivo and has a mean pore size in a range of about 75 microns to about 300 microns, about 75 microns to about 200 microns, or about 100 microns to about 200 microns, for example, at least about 75 microns, at least about 100 microns, at least about 125 microns, at least about 150 microns, or at least about 175 microns, and up to about 300 microns, up to about 275 microns, up to about 250 microns, up to about 225 microns, or up to about 300 microns. The porosity of the porous foam is at least 50% and up to about 95%, or at least about 60% and up to about 80%, for example, at least 50%, at least about 60%, at least about 65%, or at least about 70% and up to about 95%, up to about 90%, up to about 85%, or up to about 80%. In general, it is desirable that the porous foam has as many pores as possible of the appropriate pore size in order to facilitate wound healing and removal of wound exudate.

As shown in Example 1, the fibrin foam of the disclosure demonstrated increased elasticity, permeability, and porosity over fibrin sealants, as well as the PU foams generally used in NPWT treatments. Advantageously, the fibrin foam of the disclosure includes pores having a porosity in a range of about 50% to 95% and pore sizes in a range of about 75 microns to about 300 microns that facilitate cellular processes that occur in wound healing, including but not limited to angiogenesis, fibroblast proliferation, and skin regeneration, as seen in Example 2. Cell biocompatibility was evaluated on human umbilical vein endothelial cells (HUVEC), normal human dermal fibroblasts (NHDF), and normal human epidermal keratinocytes (NHEK). The cells were viable and metabolically active on and within the fibrin foam, and used the fibrin foam as a matrix to adhere and spread, thus enhancing the healing process. No such enhanced healing effect was observed with PU foam. Thus, Examples 1 and 2 demonstrate the fibrin foam of the disclosure provides an added wound healing influence over PU foam.

In another related aspect, the disclosure further provides a dressing material for NPWT, including a porous fibrin foam that can substantially conform to a shape of the wound, wherein the fibrin foam comprises a three-dimensional reaction product of fibrinogen and thrombin, is biodegradable in vivo, and does not need to be changed during the course of therapy. As used herein, a fibrin foam can “substantially conform” to a shape of the wound when the fibrin foam occupies at least 90% by volume, at least 95% by volume, or at least 98% by volume of the dead space of the wound. In embodiments, the fibrin foam is applied directly from the mixing device and can substantially conform to a shape of the wound in situ, prior to setting of the fibrin foam. In embodiments, the fibrin foam is formed and set in a shape that can substantially conform to a shape of the wound, prior to application of the foam to the wound.

The disclosure further provides a dressing material for NPWT, including a porous foam dressing that can substantially conform to a shape of the wound, wherein the porous foam is characterized by a mean pore size in a range of about 75 microns to about 300 microns. In embodiments, the porous foam is applied directly to the wound and can substantially conform to a shape of the wound in situ, prior to setting of the porous foam.

Advantageously, the porous foams and/or fibrin foams disclosed herein are conformable upon application, such that the foam can be manipulated to substantially conform to a shape of the wound. Further, once set, the foam remains substantially conformed to the shape of the wound, until the foam biodegrades. This property of conformability initially and structural integrity after setting is advantageous because it mitigates the need to re-apply the dressing over the course of therapy because the loss of structural integrity is minimized. Although the foam biodegrades over time, the foam biodegrades as the wound is closing and healing into the foam, thus maintaining the shape of the wound throughout treatment.

According to U.S. Pat. No. 8,025,650 B2, the disclosure of which is herein incorporated by reference in its entirety, and as known by a person of ordinary skill in the art, the current procedure for most NPWT dressings includes manually cutting gauze or PU foam to fit the size and the shape of the wound. As many wounds treated through NPWT have irregular shapes, volumes, and/or depths, this is a time consuming and tedious process. The current standard of care does not utilize biodegradable foams or gauze, requiring the removal and replacement of wound dressings every two to five days, or at the discretion of the clinician or wound care specialist, impeding wound healing and causing the patient pain. Using the fibrin foam disclosed in the present disclosure for NPWT offers advantageous alternatives to the conventional dressings used in NPWT in both the application and maintenance of the dressing required throughout the treatment, particularly in view of the capability of the fibrin foam to substantially conform to a shape of a wound in situ.

The fibrin foam described herein is advantageously biodegradable in vivo. It is a combination of fibrinogen, a glycoprotein in vertebrates, and thrombin, an enzyme, that interact naturally to promote blood clot formation. In conventional NPWT, the gauze or PU foam dressing is applied to the wound, then covered with a semi-permeable dressing to which a vacuum pump is attached. The negative pressure, for example about −200 mm Hg to about −75 mm Hg, is applied continuously for a period of time deemed appropriate by the clinician or wound care specialist, such as two (2), three (3), four (4), five (5), six (6), seven (7), eight (8), or nine (9) days. The period of treatment generally corresponds to the length of time between removal and changing of the dressing. Conventionally, the dressing of the wound must be removed and changed every two to five days in order to reduce the risk of bacterial infections, or for the surgeon's assessment of the wound and healing. However, the removal of the dressing impedes healing, as the treatment stimulates the ingrowth of cells into the pores of the wound dressing. When the dressing is removed, the healthy cells and tissue that have grown into the pores of the PU foam or gauze dressing must be cut, causing the patient pain and impeding the healing process. Using the disclosed fibrin foam as a wound dressing allows the dressing to remain intact in the wound until it naturally degrades over the course of about fourteen days. As demonstrated in Examples 3 and 4, the fibrin foam dressing stays in place throughout the NPWT treatment, both when the conventional NPWT protocol is followed (i.e., wherein the fibrin foam is covered by a semi-permeable dressing attached to the vacuum pump) and when the dressing is exposed directly to the negative pressure without the semi-permeable dressing attached to the vacuum pump. By not being removed throughout treatment, the fibrin foam allows angiogenesis and other cellular processes involved in wound healing to occur uninterrupted. In fact, the pore size of the fibrin foam, generally from about 100 microns to about 250 microns, as shown in FIG. 2, facilitates new cell growth, whereas pore sizes greater than 250 microns and less than 100 microns have been found relatively ineffective in this respect. The pore size of PU foam is significantly larger (e.g., about 400-600 microns) and does not promote the adherence and migration of new cells and cell proliferation into the PU foam matrix.

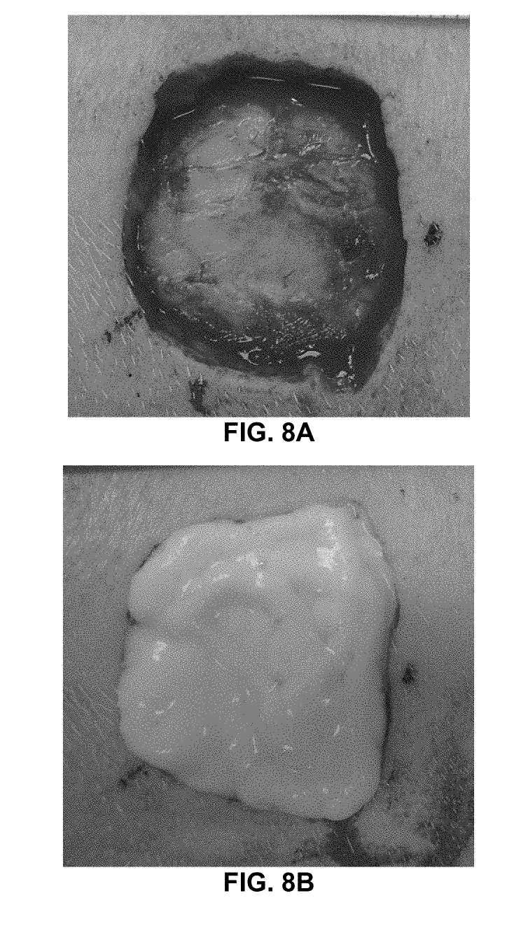

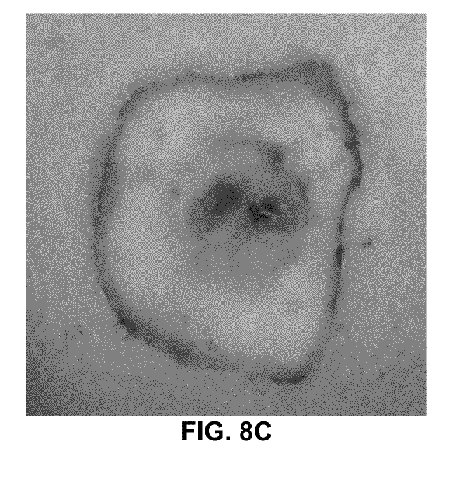

Additionally, the fibrin foam described herein has the ability to be applied directly to the wound using the mixing device. Due to the form and viscosity of the fibrin foam, the foam can be applied to vertical and inverted surfaces. Once applied, the fibrin foam can substantially conform and adapts to the shape of the wound, as shown in FIG. 8. This allows a wider range of wound shapes, volumes and/or depths to be treated more easily, without inconveniencing the patient or medical professional during the application of the dressing. In contrast, the prior art demonstrates that gauze and PU foam must be cut to the shape of the wound prior to application and cannot easily be applied to vertical and inverted surfaces.

The disclosure further provides a method of treating a wound using negative pressure wound therapy including contacting a wound with a porous fibrin foam dressing of the disclosure and applying negative pressure on the wound. In embodiments, negative pressure can be applied directly to the wound including the porous fibrin foam. In embodiments, after contacting the wound with the porous fibrin foam, the wound and fibrin foam may be covered by a semi-permeable dressing that is attached to the vacuum pump. The negative pressure can be applied at any level suitable to drain excess fluids from the wound, contract of the perimeter of the wound, reduce tissue edema, and/or mechanically stimulate the wound bed giving rise to angiogenesis and the formation of granulation tissue, as shown in FIG. 8. The negative pressure can be applied in a range of about −400 mm Hg to about −50 mm Hg, about −300 mm Hg to about −75 mm Hg, about −250 mm Hg to about −75 mm Hg, about −200 mm Hg to about −100 mm Hg, about −175 mm Hg to about −125 mm Hg, about −200 mm Hg to about −150 mm Hg, or about −125 mm Hg to about −75 mm Hg.

In embodiments, contacting the wound with a porous fibrin foam dressing includes applying a fibrin foam dressing to the wound. Optionally, contacting further includes maintaining the fibrin foam dressing in contact with the wound until the fibrin foam biodegrades.

Pore size analysis was performed on cross-sectional cuts of fibrin foam clots. SEM images were uploaded into FIJI ImageJ imaging software. Feret's diameter was measured for pores in each sample. Mean pore size was obtained for each fibrin foam preparation.

Percent porosity of fibrin foam samples was calculated using SEM images analyzed with FIJI ImageJ. Sample images were assessed for mean gray scale values over a 300×300 pixel area. Two independent measurements were taken per image with at least three images per sample.

### EXAMPLES

### Example 1—Process of Making Fibrin Foam

A fibrin foam was prepared using a 4 IU/ml thrombin solution and a 91 mg/ml fibrinogen solution from Baxter Healthcare Corporation, with no additives, in a 1:1 ratio of gas to total volume of constituents. Using a mixing device from Baxter Healthcare Corporation similar to the device shown in FIG. 1, “Syringe A” contained 1 mL of the 4 IU/ml thrombin solution plus 2 mL of gas, and “Syringe B” contained 1 mL of the 91 mg/mL fibrinogen solution. The components of Syringe A and Syringe B were passed six times through the sintered porous polyethylene disk for aeration. After 20 seconds, the fibrin foam gained its foam-like appearance and structure with a mean pore size of 155.5±58.3 microns. The foam had a porosity of 72.5±8.3%, determined by SEM, which was a 29.7% increase of porosity over the polyurethane foam. The mechanical characteristics of the fibrin foam were determined and compared to those of a fibrin sealant and a PU foam. The fibrin foam had a tensile strength of 0.40±0.07 MPa, a wound closure strength of 0.56±0.06 MPa, an elastic modulus of 0.047±0.01 MPa, and a wound closure modulus of 0.32±0.03 MPa. Tensile strength and elastic modulus were obtained using a Materials Testing System for the tensile strength test. Dogbone-shaped molds were generated for the tensile testing. Each group contained four to eight samples. The shear strength, compaction, and permeability of the resultant foam was also determined by expanding from the analysis of the TEG Analytical Software version 4.2.3. The shear strength of the fibrin foam was 3.8±0.7 kPa, compared to 3.6±1.9 kPa for the fibrin sealant. The compaction value of the fibrin foam was 24.7±1.6%, compared to 7.4±1.4% for the fibrin sealant. The permeability of the fibrin foam was (8.3±0.2)×10⁻⁸ mm², compared to (6±1)×10⁻⁸ mm² and (13±1)×10⁻⁸ mm² for the fibrin sealant and PU foam, respectively.

FIG. 2 shows the pore size distribution of the resultant fibrin foam. The typical ranges of pore size of fibrin sealants and polyurethane foam are also shown.

Thus, Example 1 demonstrates preparation of a fibrin foam according to the disclosure.

### Example 2—Use of Fibrin Foam as a Dressing in a Murine Wound Model

A fibrin foam dressing was used in a Murine Wound Model to determine its healing feasibility. Twenty-four total BKS.Cg-Dock7^{m}+/+Lepr^{db}/J mice were acquired. Seven (7) and fourteen (14) days were selected as the end points, with twelve (12) mice used per time point. The time period for full wound healing in these mice estimated to be about fourteen days, based on known reports, for example, Dobryansky et al., “Quantitative and reproducible murine model of excisional wound healing,” Wound Rep. Reg. 12 (2004), 485-492; and Gibran et al., “Validation of a model for the study of multiple wounds in the diabetic mouse (db/db),” Plastic Reconstr. Surg., 113 (2004), 953-960. Animals were placed under inhalant anesthesia and given Buprenex® pre-surgery for pain control. Four (4) full-thickness (i.e., through all skin layers down to the panniculus camosus), six (6) mm punch biopsy wounds were created on the dorsal surface of the mice. Each wound was treated separately with one control containing no dressing. The remaining three wounds were treated with experimental dressings including one with ARTISS fibrin sealant, one with fibrin foam prepared according to Example 1, and one with a polyurethane foam dressing. ARTISS fibrin sealant is a composition prepared from fibrinogen solution and thrombin solution in the absence of gas, thus providing a liquid sealant that forms a relatively non-porous material when set, rather than a foam. Tegaderm and/or cohesive tape was put on the mice to prevent access to the wound, and mice were individually caged to prevent damage by other animals to the wound site. The sites that included treatment with standard bandaging control and polyurethane dressing were changed on the “change of dressing” days: 3d, 7d, 10d, and 14d. The fibrin sealant and fibrin foam were applied once. At 7d and 14d, mice were euthanized and the wound sites were collected for analysis.

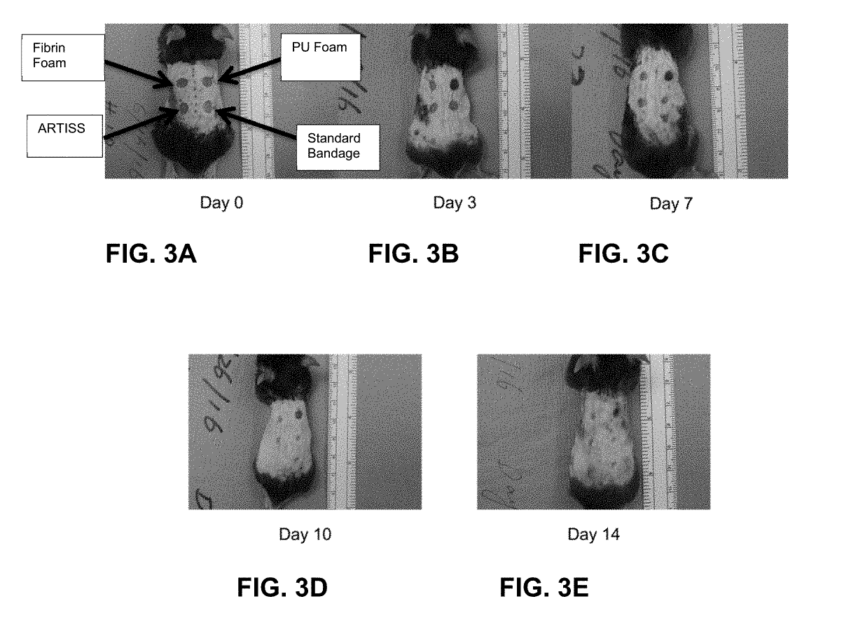

A standard camera was used for photography throughout the treatment. Images were taken before and after surgery, after treatment, and at bandage changes (3d, 7d, 10d, and 14d). FIG. 3 shows the images at Day 0 (no treatment, disclosing identifying locations where indicated treatments were applied), and at bandage changes on days 3, 7, 10, and 14. The wounds and surrounding areas were collected and plated in formalin for histopathological evaluation on change of dressing days. Histopathological evaluation was performed on the collected tissue and samples. Analysis, including hematoxylin & eosin (H&E) and Masson's Trichrome staining, were performed.

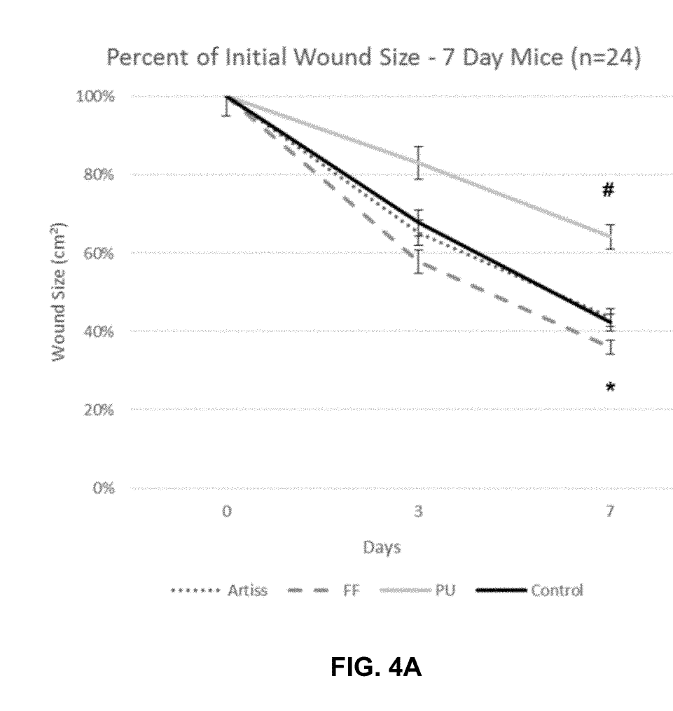

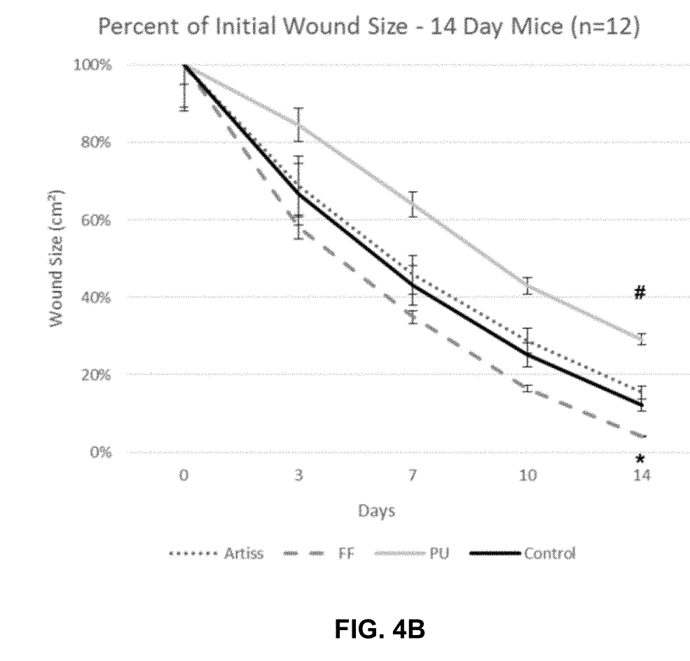

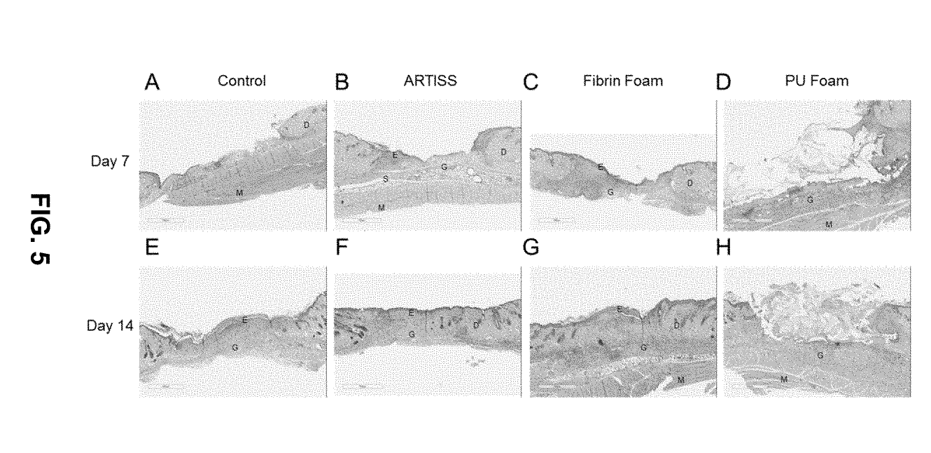

As shown in FIG. 4, the wounds treated with fibrin foam showed a significantly more rapid healing as determined by decline in mean wound size in both the 7d and 14d mice over the ARTISS fibrin sealant, PU foam, and standard-of-care bandage control treated wounds. Images from each change of dressing day were uploaded to a computer, and using imaging software (FIJI ImageJ), the wound area was measured. FIJI ImageJ imaging software was used to blindly measure the wound area by tracing the wound margin with a fine-resolution computer mouse and calculating the pixel area. Wound size area (cm²) from each day was measured as compared to day 0. A wound was considered completely closed when the wound area was equal to zero (grossly). By day fourteen, 5 (41.7%) of the wounds treated with fibrin foam had closed, compared with 2 (16.7%) of the control, 1 (8.3%) of those treated with ARTISS fibrin sealant, and 0 (0.0%) of those treated with the PU foam.

H&E and Masson's Trichrome stains were applied to assess the reepithelialization, neovascular proliferation, acute and chronic inflammation, collagen deposition, epithelial maturation, granular tissue formation, and granular tissue maturation of the wounds. As shown in FIG. 5, the fibrin foam-treated wounds healed well with no perturbations. Wounds treated with the ARTISS fibrin sealant and standard bandage control showed similar results to the fibrin foam. PU foam showed inferior wound healing, including sustained inflammation and lack of collagen deposition, when compared to all treatments.

Thus, Example 2 demonstrates treatment of a wound with a fibrin foam of the disclosure and improved treatment of a wound relative to conventional treatment processes such as a standard bandage, polyurethane foam, and ARTISS fibrin sealant.

### Example 3—Use of a Fibrin Foam Dressing in Negative Pressure Wound Therapy

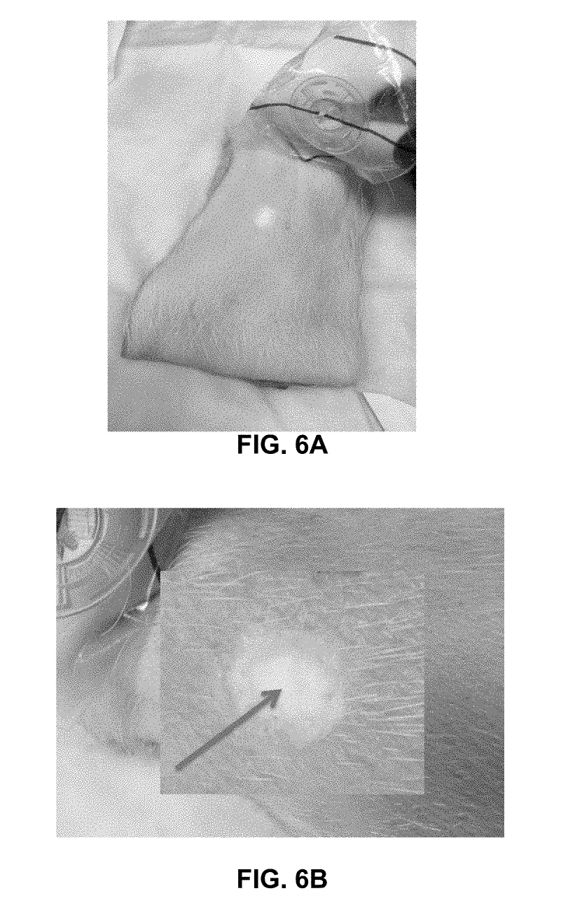

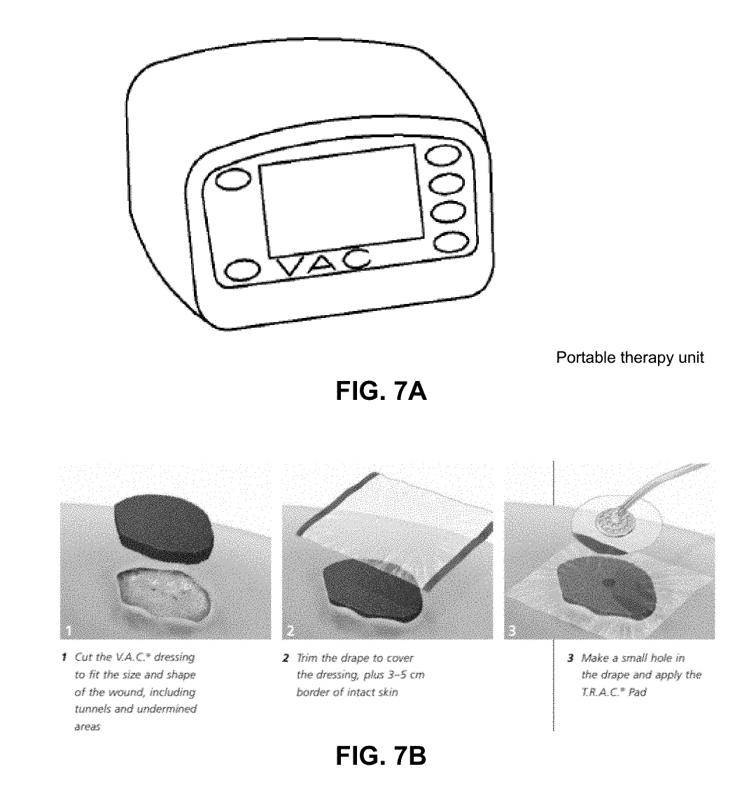

The fibrin foam prepared according to Example 1 was tested as a dressing for NPWT using V.A.C.® Freedom NPWT System from Kinetic Concepts, Inc. (KCl), as shown in FIG. 7. Fibrin foam was applied to a full-thickness, twelve (12) mm punch biopsy down to the panniculus camosus of porcine skin and allowed to cure for 5 minutes, 30 minutes, 1 hour, and 2 hours in the wound. At each time point, the NPWT system was placed over the treated wounds and −200 mm Hg pressure was applied for 2-5 minutes. After each application of negative pressure, the fibrin foam remained intact. Some of the area appeared pulled or elevated from the wound area, but no foam was pulled into the vacuum tubing system, as seen in FIG. 6.

The fibrin foam dressing was then subjected to direct pressure from the vacuum, i.e. there was no plastic sheath separating the wound dressing from the vacuum pressure, in order to demonstrate the ability of the fibrin foam to remain in the wound under extreme NPWT conditions. The fibrin foam was applied to the porcine wound and allowed to cure in the wound cavities for 30 minutes, 1 hour, and 2 hour time points prior to vacuum application. Vacuum pressure was applied directly to the wound at −200 mm Hg for 2-5 minutes. The fibrin foam remained intact.

Thus, Example 3 demonstrates the use of a fibrin foam of the disclosure in a negative pressure wound therapy treatment.

### Example 4—Use of Fibrin Foam in Negative Pressure Wound Therapy in Porcine Wound Model

The use of fibrin foam in negative pressure wound therapy (NPWT) was investigated in a Porcine Wound Model using fibrin foam without NPWT (test article 1), fibrin foam with NPWT (test article 2), a standard of care bandage (negative control), and a V.A.C.® GRANUFOAM™ Dressing with NPWT (positive control).

Materials:

Fibrin foam was prepared by separating the fibrinogen (2 mL) and thrombin (2 mL) components from the prefilled syringes of ARTISS [Fibrin Sealant (Human)] (Baxter Healthcare Corporation, Deerfield, Ill.) into individual syringes. Ambient room air (4 mL) was introduced into the thrombin syringe. The fibrinogen and thrombin syringes were then connected using a Mix-F mixing device. The Mix-F device is a dual-syringe adaptor that houses a Vyon-F porous disk (Porex, UK). The fibrinogen and thrombin solutions were passed through the Vyon-F porous disk to be aerated and mixed. The syringes constituents were manually passed back-and-forth through the Mix-F device, with one pass equaling moving through the mixing disc. Six total passes through the Mix-F device followed by a 20-second hold time were performed prior to application.

Tegaderm, or similar bandage, was applied on top of the wound packed with sterile moist gauze, and was in ready to use form.

V.A.C.® GRANUFOAM™ Dressing with NPWT (Acelity, San Antonio, Tx), a polyurethane foam dressing, was applied according to the manufacturer's instructions.

V.A.C. FREEDOM™ Therapy System for Veterinary Use was used to apply NPWT as described in the manufacturer's instructions for use. A subatomic pressure of 125 mm Hg was applied as an intermittent therapy with a 5-minute on and 2-minute off cycle. Separate wound dressings were applied to groups receiving NPWT. A single V.A.C. FREEDOM Therapy System provided subatomic pressure to both groups receiving NPWT using a Y-connector provided by the manufacturer.

Methods:

Three pigs were used for the evaluation of fibrin foam in negative pressure wound therapy. One pig was used for each of three time points (2, 5, and 8 days) to evaluate the progress of the four wound treatment types. Each animal was anesthetized and four approximately 4 cm diameter wound sites were created on the prepared dorsal thorax surface, near (approximately 3-5 cm lateral to) the midline of the animal. The wounds were measured and digitally imaged. Dressings were then applied to each of the wound sites according to Table 2:

TABLE 2

| Animal | In-Life Duration | Site | Treatment |
|---|---|---|---|
| 1 | 2 days | Cranial Left | Test 2 (Fibrin Foam with NPWT) |
| | | Cranial Right | Test 1 (Fibrin Foam without NPWT) |
| | | Caudal Left | Pos Control (Granufoam with NPWT) |
| | | Caudal Right | Neg Control (Gauze) |
| 2 | 5 days | Cranial Left | Neg Control (Gauze) |
| | | Cranial Right | Pos Control (Granufoam with NPWT) |
| | | Caudal Left | Test 2 (Fibrin Foam with NPWT) |
| | | Caudal Right | Test 1 (Fibrin Foam without NPWT) |
| 3 | 8 days | Cranial Left | Pos Control (Granufoam with NPWT) |
| | | Cranial Right | Test 1 (Fibrin Foam without NPWT) |
| | | Caudal Left | Test 2 (Fibrin Foam with NPWT) |
| | | Caudal Right | Neg Control (Gauze) |

For animal 1 (2-day time point), the dressings were managed for the in-life duration to Day 2. For animal 2 (5-day time point), the Granufoam and gauze dressings were changed on Day 2 then managed for the remaining in-life duration to Day 5. For animal 3 (8 day time point), the Granufoam and gauze dressings were changed on Day 2 and Day 5 then managed for the remaining in-life duration to Day 8. Fibrin foam with and without NPWT were not removed or replaced following application in surgery. At each bandage change or at the completion of each animal's in-life duration, the wounds were digitally imaged and measured. At the completion of each animal's in-life duration, the animals were euthanized and the wounds were harvested for histological evaluation.

Results:

All wounds were created and the treatments were successful applied. There were no procedural complications that would have affected the study objectives. All three animals treated on this study survived to the scheduled termination dates. Image analysis of the wound area and perimeter was performed for the assessment of wound size. Gross necropsy of the animals were performed and the wounds were successfully procured. Qualitative and semi-quantitative histopathological evaluation was performed to assess the biological response of the wound to the study conditions.

Endpoint 1:

Wound Size: Change in wound area after a 2-Day recovery did not show any reduction in size. Change in wound area after 5-Day recovery showed a reduction in size for all treatments except for the Negative Control (Standard of Care Bandage). During the 5-Day duration, at 2 days there was a reduction in wound size from baseline for the Granufoam Dressing and Fibrin Foam with NPWT, no change for Fibrin Foam without NPWT, and no reduction in wound size for the Negative Control. From Day 2 to Day 5 there was a small reduction in wound size for Granufoam Dressing, Fibrin Foam without NPWT, and the Negative Control. Fibrin Foam with NPWT had a larger reduction during that time period.

Change in wound area after 8-Day recovery showed a reduction in size for all treatments. During the 8-Day duration, at 2 and 5 days there was no reduction in wound size from baseline for all treatments. From Day 2 to Day 5 there was a small reduction in wound size for Fibrin Foam with NPWT, a large reduction for Fibrin Foam without NPWT, no reduction in wound size for the Negative Control, and an increase in wound size for the Granufoam Dressing. From Day 5 to Day 8 there were large reductions in wound size for all treatments.

Images of each wound were taken prior to treatment, at each bandage change and at necropsy. The Difference from Baseline, Difference from Previous Size, Percent Difference from Baseline, and Percent Difference from Previous Size were calculated for wound area only. Wound area was analyzed using Fiji software program. The results are provided in Table 3.

TABLE 3

| | | | | | | | | Percent |
|---|---|---|---|---|---|---|---|---|
| | | | | | Difference | Difference | Percent | Difference |
| | | Time- | | | from | from | Difference | from |
| | | point | Area | Perimeter | Baseline | Previous | from | Previous |
| Animal | Treatment | (Day) | (cm²) | (cm) | (cm²⁾ | Measurement | Baseline | Measurement |
| 1 | Test 2 | 0 | 11.29 | 13.02 | N/A | | | |
| | Test 1 | | 13.53 | 13.16 | | | | |
| | Pos Control | | 10.17 | 11.80 | | | | |
| | Neg Control | | 13.31 | 13.41 | | | | |
| | Test 2 | 2 | 14.91 | 14.81 | 3.62 | 3.62 | 32% | 32% |
| | Test 1 | | 16.33 | 14.55 | 2.8 | 2.8 | 21% | 21% |
| | Pos Control | | 20.18 | 16.87 | 10.01 | 10.01 | 98% | 98% |
| | Neg Control | | 14.93 | 14.16 | 1.62 | 1.62 | 12% | 12% |
| 2 | Neg Control | 0 | 11.58 | 12.19 | N/A | | | |
| | Pos Control | | 15.54 | 14.08 | | | | |
| | Test 2 | | 15.86 | 14.76 | | | | |
| | Test 1 | | 12.31 | 12.67 | | | | |
| | Neg Control | 2 | 12.60 | 12.77 | 1.02 | 1.02 | 9% | 9% |
| | Pos Control | | 14.74 | 13.94 | −0.8 | −0.8 | −5% | −5% |
| | Test 2 | | 12.58 | 13.03 | −3.28 | −3.28 | −21% | −21% |
| | Test 1 | | 12.32 | 12.78 | 0.01 | 0.01 | 0% | 0% |
| | Neg Control | 5 | 12.53 | 12.70 | 0.95 | −0.07 | 8% | −1% |
| | Pos Control | | 14.08 | 13.61 | −1.46 | −0.66 | −9% | −4% |
| | Test 2 | | 9.89 | 11.56 | −5.97 | −2.69 | −38% | −21% |
| | Test 1 | | 11.84 | 12.39 | −0.47 | −0.48 | −4% | −4% |
| 3 | Pos Control | 0 | 12.88 | 12.94 | N/A | | | |
| | Test 1 | | 11.47 | 12.23 | | | | |
| | Test 2 | | 12.12 | 12.52 | | | | |
| | Neg Control | | 12.48 | 12.75 | | | | |
| | Pos Control | 2 | 17.15 | 14.95 | 4.27 | 4.27 | 33% | 33% |
| | Test 1 | | 20.50 | 16.52 | 9.03 | 9.03 | 79% | 79% |
| | Test 2 | | 13.03 | 12.95 | 0.91 | 0.91 | 8% | 8% |
| | Neg Control | | 15.02 | 14.09 | 2.54 | 2.54 | 20% | 20% |
| | Pos Control | 5 | 34.07 | 21.08 | 21.19 | 16.92 | 165% | 99% |
| | Test 1 | | 12.02 | 12.62 | 0.55 | −8.48 | 5% | −41% |
| | Test 2 | | 12.17 | 12.78 | 0.05 | −0.86 | 0% | −7% |
| | Neg Control | | 14.97 | 14.01 | 2.49 | −0.05 | 20% | 0% |
| | Pos Control | 8 | 6.10 | 9.69 | −6.78 | −27.97 | −53% | −82% |
| | Test 1 | | 4.01 | 7.27 | −7.46 | −8.01 | −65% | −67% |
| | Test 2 | | 4.86 | 9.14 | −7.26 | −7.31 | −60% | −60% |
| | Neg Control | | 6.70 | 9.36 | −5.78 | −8.27 | −46% | −55% |

Endpoint 2: Wound Healing, Gross Necropsy Results:

Gauze:

At Day 2, the defect was >95% filled by wet cotton gauze covered and surrounded by serous fluid. At Day 5, the defect was 100% filled by tan, minimally raised, wet cotton gauze. By Day 8, the defect was 100% filled by pink and tan mottled granulation tissue with no visible test article material, covered and surrounded by serous fluid.

Granufoam with Negative Pressure Wound Therapy:

At Day 2, the defect was 100% filled by the black foam-like test article material with a slightly raised center, consistent with negative pressure application. The defect and material were minimally wet and surrounding skin dry. At Day 5, the defect was still filled by minimally raised, minimally wet black foam as previously described with dry surrounding skin. By Day 8, the defect was 100% filled by pink and tan mottled granulation tissue with central crust.

Fibrin Foam:

At Day 2, the defect was >95% filled by tan test article material, covered and surrounded by serous fluid. At Day 5, the defect was 100% filled by the previously described material, confluent with the defect margins. The defect and surrounding skin were wet. By Day 8, the defect was 100% filled by pink and tan mottled granulation tissue, covered multifocally by tan exudate.

Fibrin Foam with Negative Pressure Wound Therapy:

At Day 2, the defect was 100% filled by tan test article with a central hemorrhage (confirmed histopathologically). The defect was covered by minimal amounts of serous fluid. At Day 5, the defect was filled by tan, brown, and red and tan mottled material, slightly raised, and surrounded by wet skin on palpation. By Day 8, the defect was 100% filled by pink and tan mottled granulation tissue with a central crust. The defect and surrounding skin were covered by serous fluid.

Endpoint 2: Wound Healing, Histopathology Results:

Gauze:

Test article material was characterized by bundles of numerous birefringent cotton fibers. At Day 2, the deep layer of test article material was embedded in moderate amounts of proteinaceous fluid, fibrin, hemorrhage and inflammatory cells composed of macrophages and neutrophils. The defect was lined by large amounts of fibrin admixed with proteinaceous fluid/material and hemorrhage and the deep tissue layers were expanded and separated by edema, fibrin, hemorrhage and inflammation. Fibrosis at the cut tissue margins was minimal.

At Day 5, the defect partially filled by granulation tissue, overlaid by test article material filling the defect. The superficial margins of granulation tissue were covered by stratified squamous epithelium. Macrophages and numerous neutrophils were associated a serocellular layer, test article material, and superficial granulation tissue. Test article material was embedded in moderate amounts of amorphous proteinaceous fluid, fibrin, and a serocellular layer, admixed bacterial colonies and hemorrhage. Test article material at the granulation tissue interface was invested in granulation tissue with numerous neutrophils, lesser numbers of macrophages and multifocally multinucleated giant cells.

By Day 8, the defect completely filled by mature granulation tissue, and granulation tissue of the defect margins covered by stratified squamous epithelium. Superficially, the granulation tissue was smooth and scalloped, and exhibited a serocellular layer. Test article material (cotton gauze) was not observed with the exception of few cotton fibers embedded in granulation tissue. Inflammation was most severe within superficial tissues in closer association with the serocellular layer.

Granufoam with Negative Pressure Wound Therapy:

Test article material was characterized by numerous pieces of angular tan material. At Day 2, the deep layer of test article material was embedded in moderate amounts of amorphous proteinaceous fluid, fibrin, hemorrhage and inflammation composed of macrophages and neutrophils. The deep tissue layers of the defect were markedly expanded and separated by edema, fibrin, and hemorrhage. Macrophages and neutrophils infiltrated the interface in moderate numbers, whereas deep tissue layers markedly expanded by edema contained macrophages in lesser numbers. Fibrosis at the cut tissue margins was minimal.

At Day 5, the defect was partially filled by granulation tissue, overlaid by test article material filling the defect and expanding beyond the epidermis. The superficial margins of granulation tissue were covered by stratified squamous epithelium. Macrophages and numerous neutrophils were associated with serocellular layers, test article material, and superficial granulation tissue. The deep layer of test article material was embedded in moderate amounts of amorphous proteinaceous fluid, or a serocellular layer, admixed with numerous small bacterial colonies and multifocal hemorrhage. Test article material at the granulation tissue interface was invested in granulation tissue with numerous neutrophils, and lesser numbers of macrophages and multifocally multinucleated giant cells.

By Day 8, test article material was not observed and the defect was completely filled by mature granulation tissue, and the lateral margins of granulation tissue covered by stratified squamous epithelium. Superficially the granulation tissue was angular, irregular to scalloped and exhibited a serocellular layer with numerous bacterial colonies. Inflammation was most severe within superficial tissues in closer association with the serocellular layer.

Fibrin Foam:

Test article material was characterized by eosinophilic material containing numerous, variably sized round to oval clear spaces, or a sponge-like material. The deep layer of test article material at the tissue interface was mildly compressed, and contained or was admixed with amorphous proteinaceous fluid, fibrin, hemorrhage and inflammatory cells composed of macrophages and neutrophils. Macrophages frequently contained brightly eosinophilic cytoplasmic material interpreted as test article degradation.

At Day 2, the defect was filled by test article material and lined by large amounts of fibrin admixed with proteinaceous fluid/material, hemorrhage, inflammation. A similar mix of cells and fluid/material lined the exposed lateral tissue margin and additionally contained numerous basophilic cocci bacteria. The superficial surface of test article material was lined by a thin layer of bacteria, which multifocally infiltrated subjacent test article material. The deep tissue layers of the defect multifocally were mildly expanded and separated by edema, fibrin, hemorrhage and inflammation. Focally extensively the deep tissue—test article interface was expanded by hemorrhage. Fibrosis at the cut tissue margins was minimal.

At Day 5, the defect partially filled by variably thick granulation tissue (thicker at defect margins, thin centrally), overlaid by test article material filling the defect as a smooth layer. The superficial margins of granulation tissue were covered by stratified squamous epithelium. Macrophages and numerous neutrophils were associated with serocellular layers, test article material, and superficial granulation tissue. Subjacent granulation tissue contained few macrophages, lymphocytes and plasma cells, with multifocal few aggregates of lymphocytes. Superficially, the material was flattened, or condensed with fewer clear spaces, and lined by numerous bacterial colonies. The deep portion of material was composed of numerous irregular fragments interwoven with loose irregular granulation tissue (tissue ingrowth). The interface between test article material and granulation tissue was multifocally, frequently expanded by hemorrhage. Test article material multifocally, frequently contained large numbers of bacteria, neutrophils, and proteinaceous fluid.

By Day 8 the defect was completely filled by mature granulation tissue, and lateral tissue margins covered by stratified squamous epithelium. Superficially, the granulation tissue was smooth and scalloped, and exhibited a serocellular layer with few bacterial colonies. Inflammation of macrophages and neutrophils were increased in severity in closer association with the serocellular layer.

Fibrin Foam with Negative Pressure Wound Therapy:

Test article material was characterized by eosinophilic sponge-like material and was multifocally expanded by proteinaceous fluid. The deep layer of test article material at the tissue interface was mildly compressed, and contained or was admixed with proteinaceous fluid, fibrin, hemorrhage and inflammatory cells composed of macrophages and neutrophils. Macrophages frequently contained brightly eosinophilic cytoplasmic material interpreted as test article degradation.

At Day 2, the defect was filled by an irregular, variably thick layer of test article material. The tissue margin—test article interface was lined by fibrin admixed with proteinaceous fluid/material, hemorrhage and inflammation composed of numerous neutrophils, macrophages. This mixed material and inflammatory cell layer at the lateral margins formed a serocellular layer that additionally contained numerous basophilic cocci bacteria. The superficial surface of test article material was lined by a thin layer of bacteria, which multifocally infiltrated subjacent test article material. The deep tissue layers of the defect multifocally were mildly expanded and separated by edema, fibrin, hemorrhage, and inflammation. Focally extensively, centrally, the subcutis was a thick layer markedly expanded by edema, and the deep tissue—test article interface was expanded by marked hemorrhage. Fibrosis at the cut tissue margins was minimal.

At Day 5, the defect was partially filled by granulation tissue, overlaid by test article material filling the defect as a smooth layer. The superficial margins of granulation tissue were covered by stratified squamous epithelium. Macrophages and numerous neutrophils were associated with serocellular layers, test article material, and superficial granulation tissue. Subjacent granulation tissue contained few macrophages, lymphocytes and plasma cells, with multifocal few aggregates of lymphocytes. Granulation tissue was separated from body wall muscle layers by marked edema, fibrin and hemorrhage, and focally contained embedded necrotic collagen. The deep portion of test article material was composed of numerous irregular fragments interwoven with loose irregular granulation tissue (tissue ingrowth). The interface between test article material and granulation tissue was multifocally, frequently expanded by hemorrhage. Test article material multifocally, frequently contained large numbers of bacteria, neutrophils, and proteinaceous fluid.

By Day 8, the defect was filled by mature granulation tissue, and the granulation tissue margins covered by stratified squamous epithelium. Superficially the granulation tissue was irregular to scalloped and exhibited a serocellular layer. Centrally the superficial granulation tissue exhibited raised focus of a coagulative and lytic necrosis containing hemorrhage and numerous bacterial colonies admixed with necrosis, fibrin, proteinaceous material and inflammation. This central focus was covered by minimal amounts of compressed test article material (interpreted) admixed with bacterial colonies and necrotic debris. Inflammation was most severe in associated with the serocellular layer and central necrosis, and composed predominantly of neutrophils and macrophages.

Endpoint 2: Wound Healing, Summary:

Grossly and histopathologically, between time points and test articles, all defects healed via granulation tissue formation, with some variations in gross appearance and mild variations in histopathologic findings.

Fibrin Foam test article was grossly evident as smooth tan material, versus cotton gauze and Granufoam (black sponge). Histopathologically, test article materials were morphologically distinct, with similar tissue responses with some variability in tissue profile of the granulation bed (smooth, irregular, etc.), edema and hemorrhage, resulting to formation of a thick granulation tissue bed filling the defect by Day 8. Fibrin Foam was covered by a thin layer of bacteria at Day 2, which were histologically apparent in Granufoam or Gauze defects as well by Day 5. Bacterial colonies within the tissue sections examined appeared to be larger within Fibrin Foam filled defects, interpreted to be a result of duration of the Fibrin Foam within the defect at the later time points. Granufoam and cotton gauze were removed and replaced once or twice during the study (Day 5 animal, Day 8 animal).

At Day 5, Fibrin Foam and Fibrin Foam with negative pressure both exhibited test article degradation at the tissue interface margins, with integration of test article fragments into the developing granulation bed. The margins of Granufoam and cotton gauze were invested in granulation tissue without notable degradation, which would suggest debridement of the superficial layer of granulation tissue with removal of either of these materials at this time point. Fibrosis did not infiltrate or invest the central portions of any of the materials, test or control.

By Day 8, a granulation tissue bed filled the defects with mild variability in crusting, serous fluid coverage and exudate without moderate differences between test and control groups, but with the exception of superficial, central necrosis, associated with Fibrin Foam and negative pressure therapy. Formation of this focal, central coagulative necrosis was interpreted to be a result of direct contact of the granulation bed with the negative pressure port. After removal of the port, presumably this tissue would heal via formation of a crust and reepithelialization from the defect margins.

Conclusion:

With respect to the assessment of wound size and wound healing, over the shorter 2- or 3-day durations of observation during the study, Granufoam Dressing, Fibrin Foam with NPWT, and Fibrin Foam without NPWT showed greater reduction in wound size over the Negative Control. Over the longer 8-day duration, all treatments showed a definite reduction in wound size and displayed similar healing progression with only mild variations in histopathologic findings. Additionally, the assessment methodology indicated that Fibrin Foam with and without NPWT was degradable in the wound; and Fibrin Foam with and without NPWT yielded the same or improved wound healing relative to the Positive and Negative Controls.

Thus, Example 5 shows a method of treating a wound using negative pressure wound therapy according to the disclosure. Example 5 further shows that the biodegradable fibrin foams promote wound healing the same or better than the current standard of care polyurethane foam. Additionally, the biodegradable foams do not require debriding and reapplication over the course of healing, which is better for the patient and avoids causing additional trauma at the wound site.

The foregoing description is given for clearness of understanding only, and no unnecessary limitations should be understood therefrom, as modifications within the scope of the disclosure may be apparent to those having ordinary skill in the art.

All patents, publications, and references cited herein are hereby fully incorporated by reference. In case of a conflict between the present disclosure and incorporated patents, publications, and references, the present disclosure should control.

What is claimed:

1. A method of treating a wound using negative pressure wound therapy, the method comprising:
contacting a wound with a porous fibrin foam dressing; and
applying negative pressure on the wound,
wherein the fibrin foam comprises a three-dimensional reaction product of a gas, fibrinogen and thrombin and is biodegradable in vivo.

2. The method of claim 1, wherein the pores of the fibrin foam are characterized by a mean pore size in a range of about 75 microns to about 300 microns.

3. The method of claim 2, wherein the mean pore size is in a range of about 75 microns to about 200 microns.

4. The method of claim 1, wherein the fibrin foam has a porosity of at least 50% by volume.

5. The method of claim 4, wherein the fibrin foam has a porosity of at least 70% by volume.

6. The method of claim 1, wherein the contacting comprises applying the fibrin foam dressing to the wound and maintaining the fibrin foam dressing in contact with the wound until the fibrin foam biodegrades.

7. The method of claim 1, further comprising preparing the fibrin foam by admixing a volume of a fibrinogen solution, a volume of a thrombin solution, and a volume of gas.

8. The method of claim 7, wherein the ratio of the volume of the fibrinogen solution to the volume of thrombin solution is about 1:1.

9. The method of claim 7, wherein the ratio of the volume of gas to the sum of the volume of the fibrinogen solution and thrombin solution is in a range of about 1:1 to about 2.5:1.

10. The method of claim 9, wherein the gas comprises air.

11. The method of claim 7, wherein fibrinogen solution has a concentration of about 1 mg/ml to about 200 mg/ml fibrinogen.

12. The method of claim 11, wherein the fibrinogen solution has a concentration of about 100 mg/ml fibrinogen.

13. The method of claim 7, wherein the thrombin solution has a concentration of about 0.01 IU/m to about 500 IU/ml thrombin.

14. The method of claim 13, wherein the thrombin solution has a concentratio0n of about 4 Ul/ml thrombin.

15. The method of claim 7, wherein the fibrin foam is prepared using a mixing device comprising
at least one mixing disc having two opposing sides and comprising a three-dimensional lattice defining a plurality of tortuous, interconnecting passages therethrough;
a first container in fluid communication with one side of the mixing disc and holding the fibrinogen solution;
a second container in fluid communication with the other side of the mixing disc and holding the thrombin solution; and
each container being in fluid communication with the other container through the mixing disc to allow one of the fibrinogen solution or thrombin solution to flow from one side of the mixing disc to the other side of the mixing disc and to allow return flow of both components through the mixing disc;
wherein at least one of the first container or second container further comprises the volume of gas.

16. A dressing material for negative pressure wound therapy, comprising:
a porous fibrin foam that can substantially conform to a shape of the wound,
wherein the fibrin foam comprises a three-dimensional reaction product of fibrinogen and thrombin, is biodegradable in vivo, and does not need to be changed during the course of therapy.

17. The dressing of claim 16, wherein the pores of the fibrin foam are characterized by a mean pore size in a range of about 75 microns to about 300 microns.

18. The dressing of claim 17, wherein the mean pore size is in a range of about 75 microns to about 200 microns.

19. The dressing of claim 16, wherein the fibrin foam has a porosity of at least 50% by volume.

20. The dressing of claim 19, wherein the fibrin foam has a porosity of at least 70% by volume.

21. A method of treating a wound using negative pressure wound therapy, the method comprising:
contacting a wound with a porous foam dressing; and
applying negative pressure on the wound,
wherein the porous foam comprises pores characterized by a mean pore size in a range of about 75 microns to about 300 microns and the foam is biodegradable in vivo.

22. The method of claim 21, wherein the mean pores size is about 75 microns to about 200 microns.

23. The method or claim 21, wherein the porous foam has a porosity of at least 50% by volume.

24. The method of claim 23, wherein the porous foam has a porosity of at least 70% by volume.

25. The method of claim 21, wherein the porous foam comprises a three-dimensional reaction product of gas, fibrinogen and thrombin.

26. The method of claim 21, wherein the contacting comprises applying the porous foam dressing to the wound and maintaining the porous foam dressing in contact with the wound until the porous foam biodegrades.
